(19) United States
(12) Patent Application Publication
(10) Pub. No.: US 2001/0031221 A1
(43) Pub. Date: Oct. 18, 2001

(54) Apparatus and method for delivering fluids to contact surfaces between parts of a medical device

(76) Inventors: Su-Syin S. Wu, Irvine, CA (US); Nancy S. Chu, Laguna Niguel, CA (US); Szu-Min Lin, Laguna Hills, CA (US)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/746,990

(22) Filed: Dec. 22, 2000

Related U.S. Application Data
(63) Continuation-in-part of application No. 09/384,761, filed on Aug. 27, 1999, now Pat. No. 6,187,265.
Application No. 09/384,761 is a continuation of application No. 08/992,131, filed on Dec. 17, 1997, now abandoned.

(51) Int. Cl. A61L 2/16

(52) U.S. Cl. 422/28; 422/292

(57) ABSTRACT

An apparatus and method for delivering fluids to contact areas between parts in a medical device having two or more parts is disclosed. A plurality of projections are placed on the contact area, and, when fluid is applied to the medical device, more fluid flows around the projections than through the material from which the medical device is made. The medical device can be, for example, a scissors, a forceps, a holder, a hemostat, or a rongeur. The fluid can be a cleaning fluid, a rinsing fluid, a scrubbing fluid, or a germicide. The cleaning, rinsing, scrubbing, disinfecting, or sterilizing may be done in a vessel at ambient pressure or reduced pressure. During the contacting, more fluid flows around the projections than through the material from which the medical device is made.

### RELATED APPLICATIONS

This application is a continuation-in-part of application Ser. No. 09/384,761, filed Aug. 27, 1999, which is a continuation of application Ser. No. 08/992,131, filed Dec. 17, 1997, now abandoned.

### FIELD OF THE INVENTION

The invention relates to an apparatus and a method for effectively delivering cleaning fluid, rinsing fluid, scrubbing fluid, or germicide to contact surfaces between parts of a medical device.

### BACKGROUND OF THE INVENTION

Articles such as medical instruments are normally sterilized before use. There are many methods of sterilizing medical equipment, including heat treatment and chemical methods. Heat sterilization is normally performed with steam. Some equipment cannot withstand either the heat or the moisture from steam treatment. As a result, chemical sterilization is now commonly used.

Chemical sterilization uses a sterilizing fluid such as hydrogen peroxide, ethylene oxide, chlorine dioxide, peracetic acid, or a combination thereof. A plasma may be induced to enhance the sterilization process. Although chemical sterilization is normally highly effective, it may not be as effective with medical devices having long, narrow tubes, or lumens. It is difficult for the sterilizing agent to completely penetrate and sterilize these long narrow tubes. In order to enhance the penetration of the sterilizing agent down the entire length of the lumen, several forms of apparatus have been developed to flow sterilizing agent through the length of the lumen, enhancing the effectiveness of the sterilizing treatment.

For example, U.S. Pat. Nos. 4,410,492 and 4,337,223 describe a sterilization method in which the lumen is placed in a socket connected to a valve and a recirculating pump. The sterilizing gas is recirculated from the sterilization chamber through the lumen of the instrument. Although the method is effective at sterilizing the lumen, sterilization of endoscopes requires 2-3 hours using ethylene oxide as the sterilizing gas.

U.S. Pat. No. 5,580,530 describes a method for delivering sterilizing agent through long, narrow lumens. The lumen is inserted into an adaptor connected to a vessel containing hydrogen peroxide. The vessel is called a booster. The lumen, adaptor, and booster are placed in a sterilization chamber. When the sterilization chamber is evacuated during the sterilization procedure, the hydrogen peroxide in the booster vaporizes and passes through the lumen, sterilizing the interior of the lumen.

In each of these sterilization methods, the lumen is held by a connecting device, a socket in the case of U.S. Pat. Nos. 4,410,492 and 4,337,223 or a truncated cone adaptor when using the method of U.S. Pat. No. 5,580,530. In all of these methods, there are areas of contact between the device and the lumen in the area where the lumen attaches to the connecting device. It is difficult for the sterilizing agent to penetrate into these contact areas. There is a need for an apparatus and a method of enhancing the penetration of sterilizing gas or vapor into these contact areas more effectively to allay any potential concerns about incomplete sterilization.

There are also contact areas between the parts of medical devices having two or more pieces. It is difficult to sterilize the contact areas between the parts which make up the medical device. There is a need for a method and an apparatus for enhancing the penetration of sterilant into the contact areas between the pieces which make up the medical device.

### SUMMARY OF THE INVENTION

One aspect of the invention involves a medical device having at least two parts with contact areas between the parts. The medical device has a plurality of projections on at least one contact area. The projections and the material from which the medical device is made are adapted such that, when fluid is applied to the contact area, more fluid flows around the projections than through the material from which the medical device is made. Advantageously, at least one of the parts is movable. Preferably, at least one of the parts of the medical device is movable around a pivot.

In an embodiment, the medical device is reusable or disposable. Advantageously, the medical device includes a joint, a hinge, a box lock, or a mated surface. Preferably, the medical device is a scissors, a forceps, a holder, a hemostat, or a rongeur. In an embodiment, the medical device includes a connector housing or a luer lock. The medical device may be made of a metal or a non-metal.

Advantageously, the metal from which the medical device is made is stainless steel, titanium alloy, aluminum alloy, or nickel-chromium alloy. Preferably, the non-metal from which the medical device is made is polytetrafluoroethylene, nylon, polyolefin, liquid crystal polymer, polyester, silicon rubber, or styrenic thermoplastic. The fluid may be a cleaning fluid, a rinsing fluid, a scrubbing fluid, or a germicide. The germicide may be a liquid, gas, or vapor disinfectant or sterilant. The plurality of projections may be located randomly on the contact area or may be located in a regular pattern. The plurality of projections may be points, lines, or a combination of points and lines.

Another aspect of the invention involves a method of cleaning, rinsing, scrubbing, disinfecting, or sterilizing a medical device having at least two parts, where there is at least one contact area between the parts. The method includes having a plurality of projections on the entire contact area, contacting the medical device with a cleaning fluid, a rinsing fluid, a scrubbing fluid, a disinfecting fluid, or a sterilizing fluid, where the projections and the material from which the medical device is made are adapted such that more fluid flows around the projections than through the material from which the medical device is made.

Preferably, at least one of the parts of the medical device is moved during the cleaning, rinsing, scrubbing, or sterilizing. The medical device may be contacted with fluid in a vessel. Advantageously, the method also includes circulating the fluid in the vessel. The pressure in the vessel may be reduced to vaporize the fluid.

### BRIEF DESCRIPTION OF THE DRAWINGS

FIG. 1 is a perspective drawing of an assembled booster and adaptor with a lumen inserted in the opening of the adaptor;

FIG. 2 is an exploded perspective drawing of the booster, adaptor, and lumen of FIG. 1;

FIG. 3A is a sectional view of the adaptor and lumen, showing how the lumen fits into the opening of the adaptor;

FIG. 3B is a sectional view of the adaptor and lumen, with the lumen inserted into the opening of the adaptor;

FIG. 4 is a blow-up of FIG. 3B showing a sectional view of the area of contact between the adaptor and the lumen, where the flow of sterilant vapor through the textured area of the adaptor and through the material of the adaptor is shown with arrows;

FIG. 5 is a schematic drawing of a pair of scissors having contact areas between the two parts of the scissors;

FIG. 6A is a sectional view of the contact area of the scissors of FIG. 5 with the scissors in a closed position, where one of the pieces making up the scissors is textured, according to an embodiment of the invention;

FIG. 6B is a sectional view of the contact area of the scissors of FIG. 5 with the scissors in an open position, where one of the pieces making up the scissors is textured, according to an embodiment of the invention;

FIG. 7 is a schematic drawing of a pair of scissors having contact areas between the two parts of the scissors;

FIG. 8A is a sectional view of the contact area of the scissors of FIG. 7 with the scissors in a closed position, where both pieces of the scissors are textured, according to an embodiment of the invention;

FIG. 8B is a sectional view of the contact area of the scissors of FIG. 7 with the scissors in an open position, where both pieces of the scissors are textured, according to an embodiment of the invention;

FIG. 8C is a sectional view of the contact area of the scissors of FIG. 7 with the scissors in a closed position, where both pieces of the scissors are textured, according to an embodiment of the invention;

FIG. 8D is a sectional view of the contact area of the scissors of FIG. 7 in an open position, where both pieces of the scissors are textured, according to an embodiment of the invention;

FIG. 9 is a perspective view of a contact area between two parts of a medical device, where both parts are textured and where the two parts are in a closed position;

FIG. 10 is a perspective view of a contact area between two parts of a medical device, where both parts are textured and where the two parts are in an open position;

FIG. 11 is a schematic drawing of a pair of scissors having contact areas between the two parts of the scissors;

FIG. 12A is a perspective view of texturing according to an embodiment of the invention, where the texturing is in the form of projections placed randomly on the contact surface;

FIG. 12B is a perspective view texturing according to an embodiment of the invention, where the texturing is in the form of projections placed in rows on the contact surface; and

FIG. 12C is a perspective view of texturing according to an embodiment of the invention, where the texturing is in the form of grooves.

### DETAILED DESCRIPTION OF THE PREFERRED EMBODIMENT

The embodiments of the method and the apparatus of the present invention relate to the sterilization, disinfection, rinsing, or cleaning of articles such as medical instruments having contact surfaces. Although the embodiments of the apparatus and the method are discussed with the example of sterilizing areas of contact between a lumen and an adaptor, the apparatus and the method have broad applicability to a variety of forms of apparatus and methods. For example, the embodiments of the apparatus and the method of the present invention can be applied to disinfection, rinsing, or cleaning as well as sterilization.

The embodiments of the method and the apparatus apply to any situation in which there are contact areas between an article to be sterilized, disinfected, rinsed, or cleaned and a device, part, adaptor, external housing, or connector. The embodiments of the method and the apparatus also apply to medical devices having two or more parts, where there are points of contact between the two parts. The embodiments of the method and the apparatus can be applied wherever contact areas exist on a device. The terms “sterilize”, “sterilant”, and other forms of this word throughout the specification and claims are to be construed broadly and are to be understood to include disinfection and other antimicrobial processes.

Embodiments of the method and the apparatus of the present invention are applicable to, for example, sterilization, rinsing, disinfection, or cleaning of lumens or medical instruments having one or more lumens. The term instruments having one or more lumens as used herein applies to medical or surgical devices such as endoscopes, catheters, tubing, or similar instruments or articles having one or more internal lumens. In this embodiment of the device and the method of the present invention, antimicrobial fluid may be supplied directly to the lumen or interior of the tube of the instrument during the sterilization process. In general, the lumen is held by an adaptor which is connected to a source of antimicrobial agent or germicide. There are contact surfaces between the adaptor and the lumen.

To enhance the sterilization, rinsing, disinfection, or cleaning of the contact surfaces, one or a combination of the following properties may be utilized in the adaptor, medical device, or connector design and material selection: first, applying texture or uneven surfaces to the contact area so as to reduce surface contact and enhance axial diffusion of sterilant; second, constructing the adaptor, medical device, or connector, at least in the contact area, from a material which has minimal chemical and physical interaction with the sterilant; and third, using a material of construction, at least in the contact area, which is permeable to the sterilant so that the sterilizing agent can penetrate the material, enhancing radial diffusion of the sterilant.

The texture or uneven surfaces are designed so that more sterilant, disinfectant, rinsing fluid, or cleaning fluid can flow around the texture or the uneven surfaces on the adaptor or connector than flows though the material of the adaptor or connector.

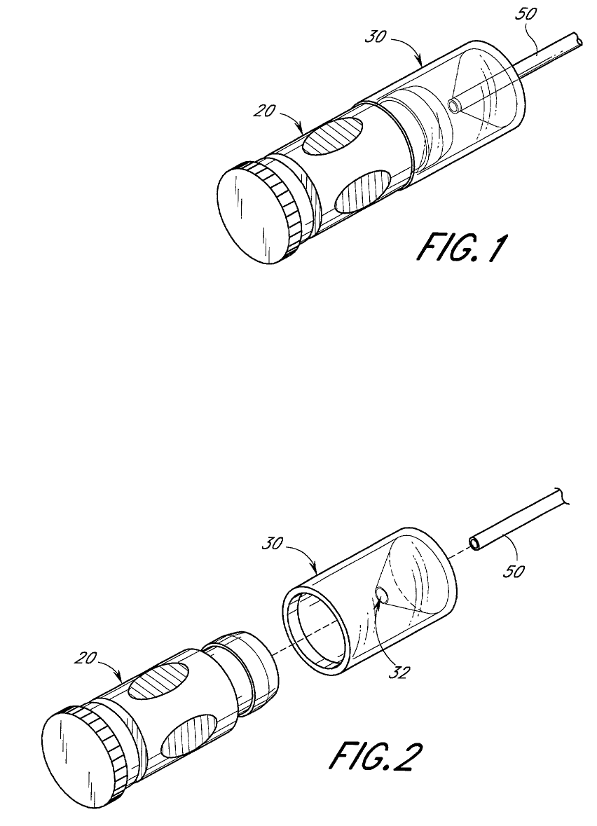

FIGS. 1 and 2 illustrate an embodiment of an apparatus suitable for use in an embodiment of sterilizing or disinfecting a lumen. FIG. 1 shows the assembled apparatus, and FIG. 2 is an exploded view, showing the various parts of the apparatus. A booster 20 is attached to an adaptor 30. A lumen 50 is inserted into an opening 32 of the adaptor 30. The opening 32 is normally of slightly smaller diameter than the outer diameter of the lumen 50 so that there is a snug fit between the inside of the opening 32 and the outside of the lumen 50.

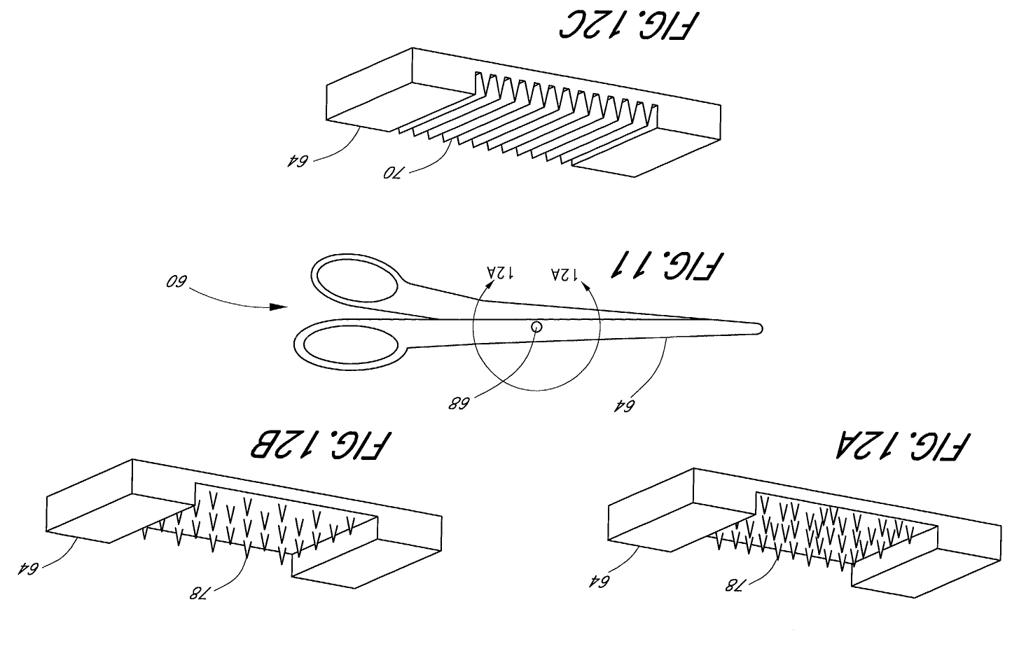

Two forms of the booster 20 are described in detail in col. 9 line 11 to col. 12, line 19 and FIGS. 5 to 13 of U.S. Pat. No. 5,580,530, hereby incorporated herein by reference in its entirety. Briefly, the booster 20 includes a vessel for containing hydrogen peroxide, a membrane wall capping the vessel containing the hydrogen peroxide, and an opener with a hollow spike which is used to breach the membrane wall, activating the booster so that the hydrogen peroxide can escape from the vessel. One form of the booster is shown as 100 on FIGS. 5 to 9 and an alternative form as 200 on FIGS. 10 and 11 of U.S. Pat. No. 5,580,530.

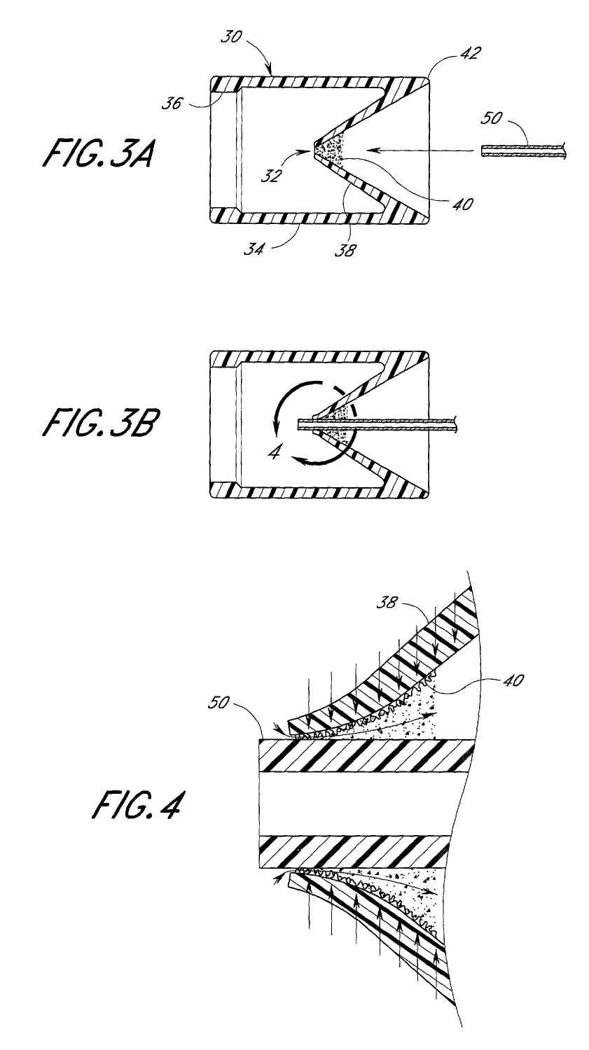

The adaptor 30 is shown in more detail in FIG. 3A herein. The adaptor 30 includes a cylindrical tubular body 34, an inwardly facing annular flange 36 for firmly attaching the cylindrical tubular body 34 to the booster 20, a truncated cone 38, the opening 32, and texturing 40 on the outer surface of the truncated cone 38 surrounding the opening 32. The adaptor has one or a combination of the following properties.

First, texturing can be added to the contact surface. The texturing can take various forms such as ridges, concentric rings, uneven surfaces, projections having equal heights, projections with varying heights, etc. Whatever form of texturing is used, there can be a plurality of the ridges, rings, or projections of equal or varying heights. The height of the texturing varies and is generally related to the viscosity of the antimicrobial or cleaning fluid. The height of the texture varies from approximately 0.0001 millimeters to approximately 50 millimeters. The height of the texture for an antimicrobial fluid which is a gas will generally be less than for an antimicrobial fluid which is a liquid, because a gas has a lower viscosity than a liquid. Although the height of the texturing can be determined by one skilled in the art, in general, a height of approximately 0.001 millimeters to approximately 5 millimeters is preferred for an antimicrobial agent which is a gas. The height of the texturing for a gas is more preferably in the range of approximately 0.01 millimeters to approximately 2.0 millimeter, and most preferably in the range of approximately 0.1 millimeters to approximately 1.0 millimeters. The height of the texturing which is preferred for a liquid is normally in the range of approximately 0.01 to approximately 5 millimeters, depending on the viscosity of the liquid. The height of the texturing for a liquid is more preferably in the range of approximately 0.1 millimeters to approximately 4 millimeters, and most preferably in the range of approximately 0.2 to approximately 2 millimeters.

The texturing preferably extends to the inside of the opening 32, so that the area directly facing the lumen 50 as well as the outer surface of the truncated cone 38 surrounding the opening 32 is textured. The portion of the truncated cone 38 which is textured is preferably in the range of approximately 0.001 to 50 millimeters, more preferably in the range of approximately 0.01 millimeters to approximately 20 millimeters, and most preferably in the range of approximately 0.1 millimeters to approximately 10 millimeters, radically extending from the edge of the opening 32. The amount of the contact area to be covered with texture may depend on the length of the occluded area. The total length of the textured surface is preferably approximately 5 times the length of the occluded area, more preferably approximately 3 times the length of the occluded area, and most preferably approximately 1.5 times the length of the occluded area. The inwardly facing annular flange 36 fits into a shallow annular groove on the booster 20 when the adaptor 30 is fitted into place on the booster, firmly attaching the adaptor 30 to the booster 20. Those of skill in the art will appreciate that the dimensions of the truncated cone 38 and the opening 32 can be varied to accommodate various types of instruments to be sterilized.

Second, the material, at least in the contact area, preferably is compatible with the sterilant or sterilization agent, that is, has minimum chemical and physical interaction with the sterilant or sterilizing agent. Chemical interaction includes chemical reaction or catalytic decomposition of the sterilant. Physical interaction includes absorption or adsorption of the sterilant by the material. Third, the material, at least in the contact area, can be permeable to the sterilant so that the antimicrobial fluid can penetrate through the material.

Suitable materials for fabricating the adaptor, at least in the contact area, can include, but are not limited to, polyolefins (including thermoplastic elastomers), fluorinated and/or chlorinated polyolefins (including thermoplastic elastomers), fluorovinylidene, chlorovinylidene, liquid crystal polymers such as wholly aromatic polyester or polyester-amide, silicone rubber, fluorinated silicone rubber, or polyester. These materials can be mixed with one or more fillers which have minimum chemical/physical interactions with the chemical sterilant. Fillers can be added to enhance mechanical, electrical, or thermomechanical properties.

The following procedure may be used when sterilizing equipment with the booster 20 and the adaptor 30. An appropriately sized adaptor 30 is selected for the particular lumen 50 or other equipment to be sterilized. The adaptor 30 is attached to the booster 20, and the lumen 50 or other instrument to be sterilized is inserted into the opening 32. The booster 20 is activated by puncturing the membrane wall, and the hydrogen peroxide or other sterilizing agent is free to enter the adaptor 30 and the interior of the lumen 50 or instrument. In general practice, the activated booster 20, adaptor 30, and lumen 50 are placed into a sterilization chamber, the chamber is sealed, and the chamber is evacuated, preferably to a pressure of approximately 100 torr or less, more preferably to a pressure of approximately 50 torr or less, and most preferably to a pressure of approximately 10 torr or less. An antimicrobial fluid is then injected into the chamber, where it vaporizes and contacts the exposed surface of the equipment. Various factors known to those skilled in the art can be used to enhance sterilization such as heat, plasma, or high frequency radiation.

The hydrogen peroxide or other antimicrobial fluid in the booster 20 volatilizes when the chamber is evacuated. The germicide vapor enters the adaptor 30 and the lumen 50, sterilizing the interior of the lumen. The exterior of the lumen is sterilized by the antimicrobial agent which is injected into the chamber.

FIGS. 3A and 3B illustrate the use of the adaptor 30 with a lumen 50. One skilled in the art can appreciate that the size of the opening 32 on the adaptor 30 can be varied, depending on the size of the lumen 50 or other equipment connected to the adaptor 30. The body of the adaptor 30 can have shapes other than a cylinder, depending on the shape of the booster 20. For example, a rectangular adaptor 30 would be used if the booster 20 were rectangular. Similar modifications would be obvious to those skilled in the art.

The adaptor 30 can have several features which make the sterilization of the lumen 50 even more effective than previous devices. Some of these features are illustrated in FIG. 4, which is a blowup of FIG. 3B, showing the area of contact between the lumen 50 and the adaptor 30. First, the areas of contact between the adaptor 30 and the lumen 50 or other medical device can be reduced by using textured surfaces on the adaptor 30. Thus, the opening 32 and the part of the truncated cone 38 which contact the lumen 50 can be textured, as shown in FIG. 4. Only the tips of the texturing devices remain as areas of contact between the adaptor 30 and the lumen 50. The contact area is far less than if the texturing were not present. In addition, there are small gaps between the ridges or “bumps” of the texturing which create an uneven surface. The uneven surface allows fluid penetration in both longitudinal and transverse directions. Therefore, the antimicrobial agent, rinsing fluid, or cleaning fluid can enter these gaps and reach areas which would otherwise be inaccessible.

Finally, if the material used to construct the adaptor 30 is permeable to the antimicrobial agent, typically hydrogen peroxide, peracetic acid, or chlorine dioxide, further enhancement of the sterilization effectiveness can be achieved. The antimicrobial agent can penetrate the adaptor 30 to reach any areas of contact between the adaptor 30 and the lumen 50 or other instrument which remain after the contact areas are minimized through surface texturing. FIG. 4 shows arrows illustrating the penetration of the sterilant vapor to the contact areas both through the gaps between the unevenness of the texturing and through the permeable material from which the adaptor 30 can be fabricated.

The effectiveness of penetration of the antimicrobial agent through the material of the adaptor 30 to the contact areas can be even further enhanced by making the adaptor 30 thinner in the contact areas than in the remainder of the adaptor 30. For example, in FIGS. 3A and 4, the wall thickness of the truncated cone 38 of the adaptor 30 decreases from the outer end 42 to the opening 32. The portion of the truncated cone 38 which is in contact with the lumen 50 is the thinnest part of the truncated cone 38, and the antimicrobial agent can penetrate to the contact area between the adaptor 30 and the lumen 50 more effectively than if the adaptor 30 in this area were thicker. Making the adaptor 30 thinner in the contact areas than in the remainder of the adaptor 30 is a way to further enhance the penetration of the antimicrobial agent through the material of the adaptor 30 into the contact area. Although this is a preferred embodiment, it is not a required feature.

By using one or a combination of these features in the adaptor 30, the antimicrobial agent can penetrate the areas of contact between the adaptor 30 and the lumen 50 more effectively than in previous designs. These features include: applying texture or uneven surfaces to the contact area so as to reduce surface contact and enhance bidirectional diffusion of sterilant; using a material which has minimal chemical and physical interaction with the sterilant; and forming the adaptor 30 from a material that is permeable to the sterilant so that the sterilizing agent can penetrate the material.

The embodiments of the method and the apparatus of the present invention can be used whenever there are areas of contact between an article to be sterilized through sterilization and a connecting device for the article. Often, the connecting device will have an aperture through which the article is inserted. There are areas of contact between the aperture of the connecting device and the article to be sterilized. The article to be sterilized can include a lumen, rod, or other device. The methods of the present invention can be used in the connecting device and/or the article to be sterilized. These methods include the use of texturing on the areas of the connecting device which contact the device to be sterilized in order to reduce the contact area between the article and the connecting device. Second, the connecting device can be made of a material which is permeable to the antimicrobial agent so that any remaining contact surfaces can be sterilized by penetration of the antimicrobial agent through the material of the adaptor. Third, the selected material can be a material which has minimal physical and chemical interaction with the antimicrobial agent. Ways to optimize these design modifications will be apparent to those skilled in the art. Generally, the height of the texturing is selected to match the viscosity of the sterilant or sterilizing agent so that more sterilant or cleaning fluid flows around the texturing than through the material of the adaptor, connector, or device. The embodiments of the method and the apparatus are applicable to sterilization, rinsing, disinfection, and cleaning of devices with contact areas.

Embodiments of the method and the apparatus of the present invention can also be used to enhance the penetration of antimicrobial agents, disinfection fluids, rinsing fluids, or cleaning fluids to contact areas within a medical device during cleaning, rinsing, disinfecting, and sterilization processes. The embodiments of the method and the apparatus have broad applicability.

Often a medical device is made of two or more pieces. There are likely to be contact areas between the pieces from which the medical device is formed. FIG. 5 shows one example of a medical device made up of two or more pieces and having contact areas, a pair of scissors 60. The pair of scissors 60 is made up of two cutting blades 64 joined at the center by a pin 68 which forms a pivot point. The portion of the cutting blades 64 in the area of the pin 68 form a contact area which is difficult to clean, disinfect, rinse, or sterilize.

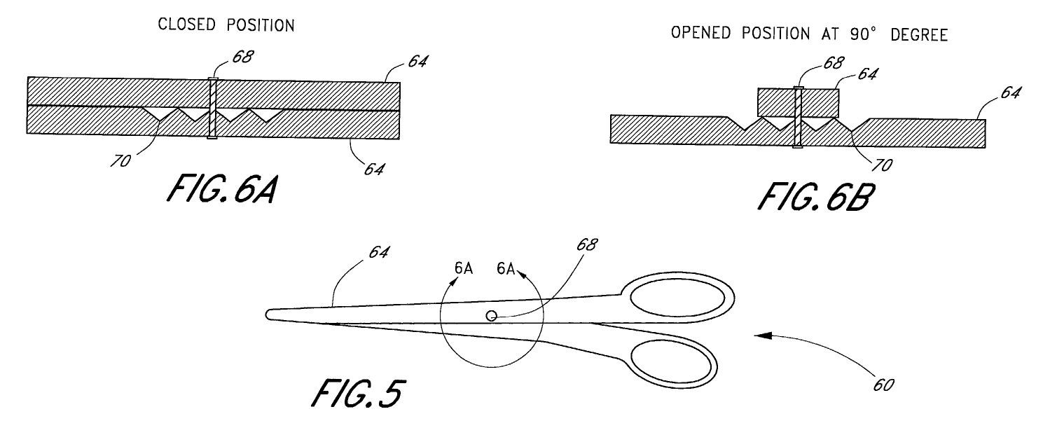

FIG. 6A shows a cross section of the two blades 64 and the pin 68 of the scissors 60 of FIG. 5, where the pair of scissors 60 is in a closed position. In the embodiment shown in FIG. 6A, a plurality of grooves 70 are present in the contact area around the pin 68 in one of the blades 64. The grooves 70 allow cleaning fluid, disinfecting fluid, rinsing fluid, or germicide to flow into the contact area, cleaning, disinfecting, rinsing, or sterilizing the contact area. FIG. 6B shows the two blades 64 of the scissors 60 in an open position. The contact area between the two blades 64 when the pair of scissors 60 is in the open position shown in FIG. 6B is less than the contact area between the two blades 64 when the scissors 60 are in the closed position, as shown in FIG. 6A. The grooves 70 allow cleaning fluid, disinfectant, rinsing fluid, or sterilant to flow into the contact areas, whether the pair of scissors 60 is in the open position or in the closed position. Because the contact area of the pair of scissors 60 is reduced when the pair is scissors 60 is in the open position, it is preferred that the cleaning, disinfecting, rinsing, or sterilizing be performed when the pair of scissors 60 is in the open position, though the grooves 70 or other texturing devices in the contact area increase the effectiveness of the cleaning, disinfecting, rinsing, or sterilizing whether the pair of scissors 60 is in the open position or in the closed position.

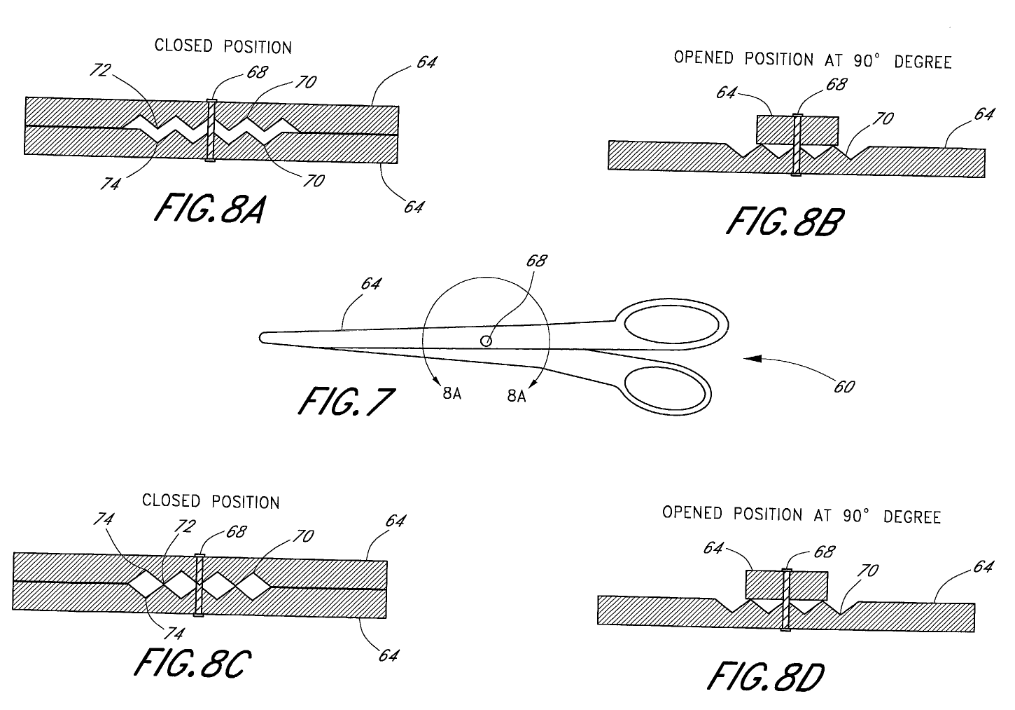

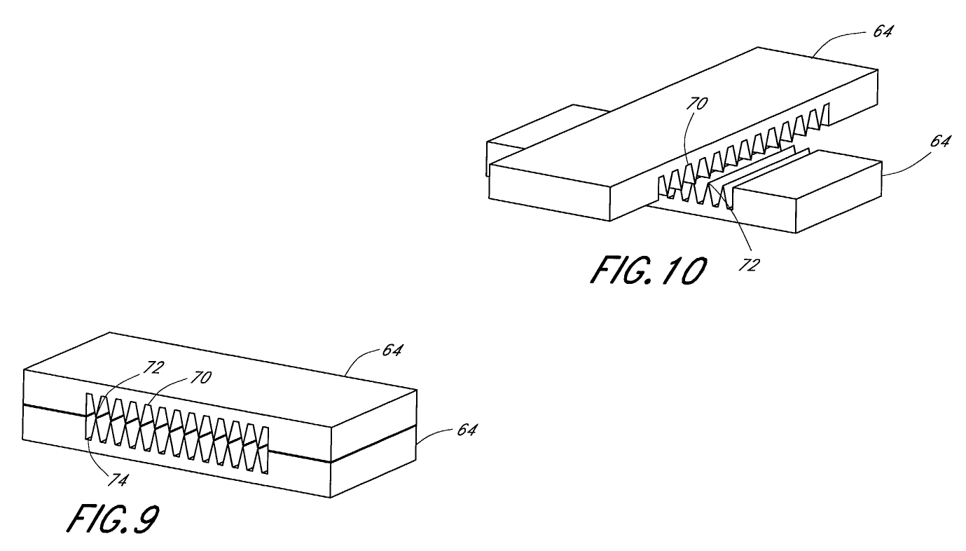

FIG. 8A shows a cross section of an embodiment of the scissors 60 of FIG. 7 in which both blades 64 making up the scissors 60 have a plurality of grooves 70 in the contact area in the region of the pin 68 which joins the two blades 64 at a pivot point. In FIG. 8A, the scissors 60 are in a closed position. FIG. 8B shows a cross section of the scissors 60 of FIG. 7 in an open position. The amount of contact area between the blades 64 in the open position shown in FIG. 8B is reduced from the contact area between the blades 64 in the closed position shown in FIG. 8A. Cleaning fluid, disinfectant, rinsing fluid, or germicide can flow through the grooves 70 into the contact area, cleaning, disinfecting, rinsing, or sterilizing the remaining contact area.

In the embodiment shown in FIG. 8A, the grooves 70 in the two blades 64 are in a staggered arrangement, that is, a point 72 of the groove 70 in an upper blade 64 is aligned with a valley 74 in a lower blade 64. As seen in FIG. 8A, there are no points of contact between the top blade 64 and the bottom blade 64 in the portion of blades 64 with grooves 70 when the blades 64 are in the closed position in the embodiment where the grooves 70 in the two blades 64 are in a staggered arrangement.

FIGS. 8C and 8D show an alternate embodiment of the scissors 60 in which the points 72 in the upper blade 64 are aligned with the points 72 in the lower blade 64, and the valleys 74 in the upper blade 64 are aligned with the valleys 74 in the lower blade 64.

FIGS. 9 and 10 show two alternative perspective views of the blades 64 of the embodiments shown in FIGS. 8C and 8D. The points 72 of the grooves 70 in a the top blade 70 are aligned with the points 72 of the grooves 70 in the bottom blade 70. In the closed position shown in FIG. 9, the contact areas between the two blades 64 are a plurality of parallel lines formed by the contact between the points 72 in the upper blade 64 and the points 72 in the lower blade 64.

FIG. 10 shows the two blades 64 in an open position. When the blades 64 are in the open position shown in FIG. 10, the areas of contact between the points 72 of the grooves 70 in the top blade 64 and the points 72 of the grooves 70 on the lower blade 64 are a plurality of points. The grooves 70 on the blades 64 thus greatly reduce the amount of contact area between the two blades 64, whether the blades 64 are in an open position or in a closed position. Because the contact areas between the blades 64 are a plurality of points when the blades 64 are in an open position versus a series of lines when the blades 64 are in a closed position, it is preferred that the blades 64 be in an open position when the cleaning, disinfecting, rinsing, or sterilization is performed. Regardless of whether the blades 64 are in an open position or in a closed position, cleaning fluid, rinsing fluid, disinfectant, or germicide can flow through the grooves 70 to clean, rinse, disinfect, or sterilize the blades 64, even the contact areas between the blades 64.

FIGS. 12A, 12B, and 12C show various embodiments of texturing that may be used to reduce the contact area between two or more parts of a medical device, for example the pair of scissors 60 shown in FIG. 11. In the embodiment shown in FIG. 12A, the texturing on the contact surface is in the form of a plurality of projections 78 in random positions on the contact surface. In the embodiment shown in FIG. 12B, the texturing on the contact surface is in the form of projections 78 aligned in regular rows on the contact surface. In the embodiment shown in FIG. 12C, the texturing on the contact surface is in the form of grooves 70. Although the projections 78 and grooves 70 of FIGS. 12A, 12B, and 12C are shown as having equal heights, in other embodiments, the projections 78 and grooves 70 can have unequal heights. Other forms of texturing on the contact surfaces are suitable for use in the embodiments of the apparatus and the method of the invention, and the embodiments of texturing shown in FIGS. 12A, 12B and 12C are not meant to be limiting.

In other embodiments, the plurality of projections 78 can have the shapes of points, lines, or a combination of points and lines. In some embodiments, the plurality of projections 78 can be combinations of the random arrangement of projections 78 of FIG. 12A, the arrangement of projections 78 in rows of FIG. 12B, and/or the grooves 70 of FIG. 12C.

The plurality of projections or texturing on the contact areas between the two or more parts surfaces provide a pathway for the cleaning fluid, rinsing fluid, scrubbing fluid, or germicide to contact the contact surfaces. The projections 78 are adapted so that when fluid is applied to the medical device, more fluid flows around the projections or texturing than through the material of which the medical device is made. The fluids can be liquid, vapor, or gas.

When medical devices are made of two or more parts with contact areas between the parts, the parts are often movable. As shown in the example of the scissors 60 of FIGS. 5, 7, and 11, the two parts are often movable around a pivot. The pivot in the example of the scissors 60 of FIGS. 5, 7, and 11 is the pin 68.

The medical device with two or more parts can be made from a variety of materials such as metal or nonmetals, including, but not limited to, TEFLON™, a tradename for polytetrafluoroethylene, nylon, a generic name for polyamide, polyolefins (including polyethylene, polypropylene, and thermoplastic elastomers), stainless steel, titanium alloy, aluminum alloy, nickel-chrome alloy, liquid crystal polymer, polyester, silicon rubbers, and styrenic thermoplastic, including thermoplastic elastomers. Further, the materials from which the two or more parts are formed need not be the same. For example, one part of the medical device can be made of metal and another part from a non-metal.

The medical device with two or more parts can be disposable or reusable. The contact areas on the medical device can be due to a joint, a hinge, a box lock, or a mated surface. Devices with hinged surfaces include scissors, forceps, and clips. Typical medical devices with two or more parts having contact surfaces include scissors, forceps, holders, hemostats, or rongeurs. The embodiments of the apparatus and the method of the present invention can also be applied to luer locks, connector housings, or any connectors that join two devices, for example, venting caps for flexible endoscopes or connectors on flexible endoscope heads for all-channel irrigators.

Fluids which may be used with the embodiments of the apparatus and the method of the invention include cleaning fluids, rinsing fluids, scrubbing fluids, or germicides. The germicide may be a liquid, a gas, or a vapor. The germicide can be a disinfectant or a sterilant.

One or more of the pieces forming the medical device can incorporate the features of the embodiments of the method or the apparatus of the present invention to enhance the penetration of the fluid to the contact areas. These features include the use of texturing or uneven surfaces on one or more of the pieces forming the medical device in the contact areas between the two or more pieces. The texturing helps to reduce the contact area. between the pieces forming the medical device. Second, one or more of the pieces forming the medical device, at least in the contact area, can be made of a material which is permeable to the antimicrobial agent. Third, the material selected to form one or more of the pieces forming the medical device, at least in the contact area, can be a material which has minimal physical and chemical interaction with the antimicrobial agent. Any one or a combination of these features can be used to enhance the penetration of the cleaning fluid, rinsing fluid, scrubbing fluid, disinfecting fluid, or sterilizing fluid to the contact areas between the two or more pieces forming a medical device.

The antimicrobials used with the embodiments of the method and devices of the various embodiments of the present invention include solutions of glutaraldehyde, hydrogen peroxide, chlorine dioxide, peracetic acid, or other antimicrobials, either in a pure form or in an inert medium. Although high concentrations of the antimicrobial agents are more effective, material compatibility and handling problems may arise at high concentrations.

When a medical device with two or more parts having embodiments of the apparatus of the present invention is cleaned, rinsed, scrubbed, disinfected, or sterilized with a liquid, the medical device is contacted with the cleaning, rinsing, scrubbing, disinfecting, or sterilizing liquid. Advantageously, the medical device is contacted with the liquid in a vessel. If the contacting is in a vessel, the liquid may be circulated in the vessel. The cleaning, rinsing, scrubbing, disinfecting, or sterilizing liquid penetrates to the contact areas of the medical device. More liquid flows around the plurality of projections on the contact surface than through the material of the medical device, thus cleaning, rinsing, scrubbing, disinfecting, or sterilizing the medical device and the contact areas between the two or more parts of the medical device. The effectiveness of the cleaning, rinsing, scrubbing, disinfecting, or sterilizing can be enhanced even further by moving the two or more parts of the medical device during the cleaning, rinsing, scrubbing, disinfecting, or sterilizing. Moving the parts of the medical device changes the contact areas between the two or more parts.

If the medical device with two or more parts having embodiments of the apparatus of the present invention is to be cleaned, rinsed, scrubbed, disinfected, or sterilized with a vapor or gas, the medical device is placed in a chamber, the chamber is sealed, and the cleaning, rinsing, scrubbing, disinfecting, or sterilizing fluid is introduced into the chamber. The pressure in the chamber may optionally be reduced to vaporize the fluid. More fluid flows around the projections on the contact area than flows though the material of the medical device to clean, rinse, scrub, disinfect, or sterilize the contact area between the two or more parts of the medical device. Contacting the medical device also cleans, rinses, scrubs, disinfects, or sterilizes the remainder of the medical device which does not have contact areas.

Various modifications and alterations of this invention will be apparent to those skilled in the art without departing from the scope and spirit of this invention. It should be understood that the invention is not limited to the embodiments disclosed therein, and that the claims should be interpreted as broadly as the prior art allows.

What is claimed is:

1. A medical device comprising at least two parts with at least one contact area therebetween, said medical device being made up of at least one material, said medical device comprising a plurality of projections on at least one contact area, wherein said projections and said at least one material are adapted such that, when fluid is applied thereto, more fluid flows around said projections than through said at least one material of said medical device.

2. The medical device of claim 1, wherein at least one of the at least two parts is movable.

3. The medical device of claim 2, wherein said at least one of the at least two parts is movable around a pivot.

4. The medical device of claim 1, wherein the medical device is reusable or disposable.

5. The medical device of claim 1, wherein the medical device comprises a part selected from the group consisting of a joint, a hinge, a box lock, and a mated surface.

6. The medical device of claim 1, wherein the medical device is selected from the group consisting of a scissors, a forceps, a holder, a hemostat, and a rongeur.

7. The medical device of claim 1, wherein said medical device comprises a connector housing or a luer lock.

8. The medical device of claim 1, wherein said at least one material is a metal or a non-metal.

9. The medical device of claim 8, wherein said metal is selected from the group consisting of stainless steel, titanium alloy, aluminum alloy, and nickel-chromium alloy.

10. The medical device of claim 8, wherein said non-metal is selected from the group consisting of polytetrafluoroethylene, nylon, polyolefin, liquid crystal polymer, polyester, silicon rubber, and styrenic thermoplastic.

11. The medical device of claim 1, wherein said fluid is a fluid selected from the group consisting of a cleaning fluid, a rinsing fluid, a scrubbing fluid, and a germicide.

12. The medical device of claim 11, wherein said germicide comprises at least one liquid, gas, or vapor disinfectant or sterilant.

13. The medical device of claim 1, wherein said plurality of projections are located randomly on said contact area.

14. The medical device of claim 1, wherein said plurality of projections are located on said contact area in a regular pattern.

15. The medical device of claim 1, wherein said plurality of projections have a shape selected from the group consisting of points, lines, and a combination of points and lines.

16. A method of cleaning, rinsing, scrubbing, disinfecting, or sterilizing a medical device having at least two parts, wherein there is at least one contact area between the at least two parts, and wherein said at least one contact area is made of at least one material, said method comprising;
providing a plurality of projections on the entire at least one contact area;
contacting said medical device with a fluid selected from the group consisting of a cleaning fluid, a rinsing fluid, a scrubbing fluid, a disinfecting fluid, and a sterilizing fluid, wherein said projections and said at least one material are adapted such that more fluid flows around said projections than through said at least one material; and
cleaning, rinsing, scrubbing, disinfecting, or sterilizing said medical device.

17. The method of claim 16, further comprising moving at least one of said at least two parts of said medical device during said cleaning, rinsing, scrubbing, or sterilizing said medical device.

18. The method of claim 16, further comprising contacting said medical device with said fluid in a vessel.

19. The method of claim 18, further comprising circulating said fluid in said vessel.

20. The method of claim 18, further comprising reducing a pressure in said vessel, thereby vaporizing said fluid.
